(19) United States
(12) Patent Application Publication
(10) Pub. No.: US 2016/0100808 A1
(43) Pub. Date: Apr. 14, 2016

(54) SYSTEM AND METHOD FOR RESPIRATORY SYSTEM ASSESSMENT

(71) Applicant: Keivan Anbarani, North Vancouver (CA)

(72) Inventor: Keivan Anbarani, North Vancouver (CA)

(21) Appl. No.: 14/510,043

(22) Filed: Oct. 8, 2014

(51) Int. Cl. A61B 5/00; A61B 5/1455; A61B 5/0205; A61B 5/113

(52) U.S. Cl. CPC A61B 5/7425; A61B 5/113; A61B 5/14552; A61B 5/0205; A61B 5/7246; A61B 5/7282; A61B 5/7445; A61B 5/087

(57) ABSTRACT

A system and method comprise sampling a rate of airflow in a patients lungs during inspiration and/or expiration of the patient and outputting a corresponding airflow signal. Respiratory muscles signals of the patient are simultaneously sampled with the sampling of the airflow and outputted as a corresponding activity signal. The airflow signal and the activity signal are simultaneously displayed wherein a comparison of at least the airflow signal and the activity signal indicates physical properties of a respiratory system of the patient.

### CROSS-REFERENCE TO RELATED APPLICATIONS

Not applicable.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to respiratory system assessment. More particularly, the invention relates to respiratory system assessment using measurement of input and output of respiratory system.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Currently available methods for assessing different aspect of respiratory systems may include spirometer and electromyography. Spirometer may be used to monitor airflow during respiration and plotting it as a pneumotachograph. Current gold standard for diagnosing a respiratory problem using a spirometer is to calculate forced/maximum expiratory volume in one second (FEV1), and divide that by forced vital capacity (FVC), which is the total expired volume of air during attempted forced expiration. Comparing patients FEV1/FVC, also called Tiffeneau-Pinelli index value, with statistically calculated FEV1/FVC values from general population, may distinguish whether a patient has an obstructive or restrictive respiratory disorder. In some of these systems, useful results may only be detectible during advanced stages of diseases when clear statistical deviation may be seen. Also, accurate results highly depend on patient cooperation and effort and may as a result be unsuitable for use on patients having limited comprehension, such as children, patients with intellectual disabilities, and unconscious or heavily sedated patients. Further, some systems may have a risk of lung collapse in patients with compromised lung airways due to pressure build-up during forced expiration. Electromyography (EMG), and particularly non-invasive electromyography, has recently been shown to be useful to measure respiratory muscle activity.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. One such aspect of the prior art shows a device and method for detecting and treating airflow limitations in a subject. By way of educational background, another aspect of the prior art generally useful to be aware of teaches of a system and method for discrimination of central and obstructive disordered breathing events. Another such aspect of the prior art discloses a system and method of monitoring respiratory airflow and oxygen concentration. However, these solutions may not be suitable for combining principles of EMG and airflow sensor to simultaneously measure respiratory system input (muscle activity) and output (airflow) to formulate information about respiratory system condition. A solution which did so would be desirable.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1A is an illustration of an exemplary graph showing effect of a patient's effort on measurement of patient's respiratory system;

FIG. 1B is an illustration of an exemplary graph showing differences between results of healthy patients and patients having various disorders as shown in separate graphs;

FIG. 2 is an illustration of an exemplary graph showing differences between results of healthy patients and patients having various disorders;

FIG. 3 is an illustration of an exemplary system in which a respiratory unit receives input and produces output, in accordance with an embodiment of the present invention;

FIG. 4 is an illustration of an exemplary system for assessing respiratory system condition, in accordance with an embodiment of the present invention;

FIG. 5 is an illustration of an exemplary method for assessing respiratory system condition, in accordance with an embodiment of the present invention;

FIG. 6 is an illustration of an exemplary simplified system for assessing respiratory system condition, in accordance with an embodiment of the present invention;

FIG. 7 is an illustration of an exemplary system for assessing respiratory system condition, having additional components, in accordance with an embodiment of the present invention;

FIG. 8 is an illustration of exemplary placement of electrodes 20, in accordance with an embodiment of the present invention;

FIG. 9 is an illustration of an exemplary graph measuring airflow of a respiratory system, in accordance with an embodiment of the present invention;

FIG. 10 is an illustration of an exemplary graph of respiratory cycles showing volume flow, acceleration, and all EMG amplitudes in one graph, in accordance with an embodiment of the present invention;

FIG. 11 is an illustration of an exemplary graph of respiratory cycles showing volume flow, acceleration, and EMG amplitudes as wells as using a short-time Fourier transform to show perspective time-frequency response associated with each muscle, in accordance with an embodiment of the present invention;

FIG. 12 is an illustration of an exemplary graph of respiratory cycles showing volume flow, acceleration, and all EMG amplitudes in one graph, and in which respiratory cycles have been measured in a simulated obstructive condition (valve partially closed), in accordance with an embodiment of the present invention;

FIG. 13 is an illustration of an exemplary graph of respiratory cycles showing volume flow, acceleration, and EMG amplitudes as wells as using a short-time Fourier transform to show perspective time-frequency response associated with each muscle, and in which respiratory cycles have been measured in a simulated obstructive condition (valve partially closed) in accordance with an embodiment of the present invention;

FIG. 14 is a block diagram depicting an exemplary client/server system which may be used by an exemplary web-enabled/networked embodiment of the present invention; and

FIG. 15 illustrates a block diagram depicting a conventional client/server communication system.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, Fire Fox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Some embodiments of the present invention may provide means and/or methods for measuring a person's respiratory system. In some of these embodiments, a system may be suitable for measuring and/or recording an input and/or output of a respiratory system. In a non-limiting example, input may be muscle activity and output may be air entering and exiting lungs. Some embodiments may combine multiple measurement methods, including, without limitation, electromyography and airflow sensor. In one or more embodiments, recorded input and/or output may be compared to formulate information regarding respiratory system condition.

Some currently available methods of respiratory system measurement may only be suitable for producing useful results during advanced stages of disease when clear statistical distinction may be seen. Some of these methods may have limited ability to detect problems and recommend treatment options. In contrast, many embodiments of the present invention may provide results which may be quantified and used for early and/or improved detection of respiratory disorders, which may in some instance result in earlier commencement of treatments and better treatment outcomes, as well as improved patient education.

Other currently available methods of respiratory system measurement may depend on patient cooperation to produce accurate results and as such may be difficult to use for patients with limited comprehension such as young children, patients with intellectual disabilities, and unconscious or heavily sedated patients. In contrast, some embodiments of the present invention may not be dependent of patient cooperation to produce results. Some of these embodiments may produce effective results using a patient's breath. In a non-limiting example, if a patient doesn't blow/breathe hard enough, less muscle activity may be produced and as a result less flow may be produced.

Still other currently available methods of respiratory system measurement may have a risk of causing lung collapse in patients with compromised lung airways due to pressure build-up during forced expiration. In contrast, some embodiments of the present invention may not use forced expiration and as such may not have a risk of causing lung collapse in patients.

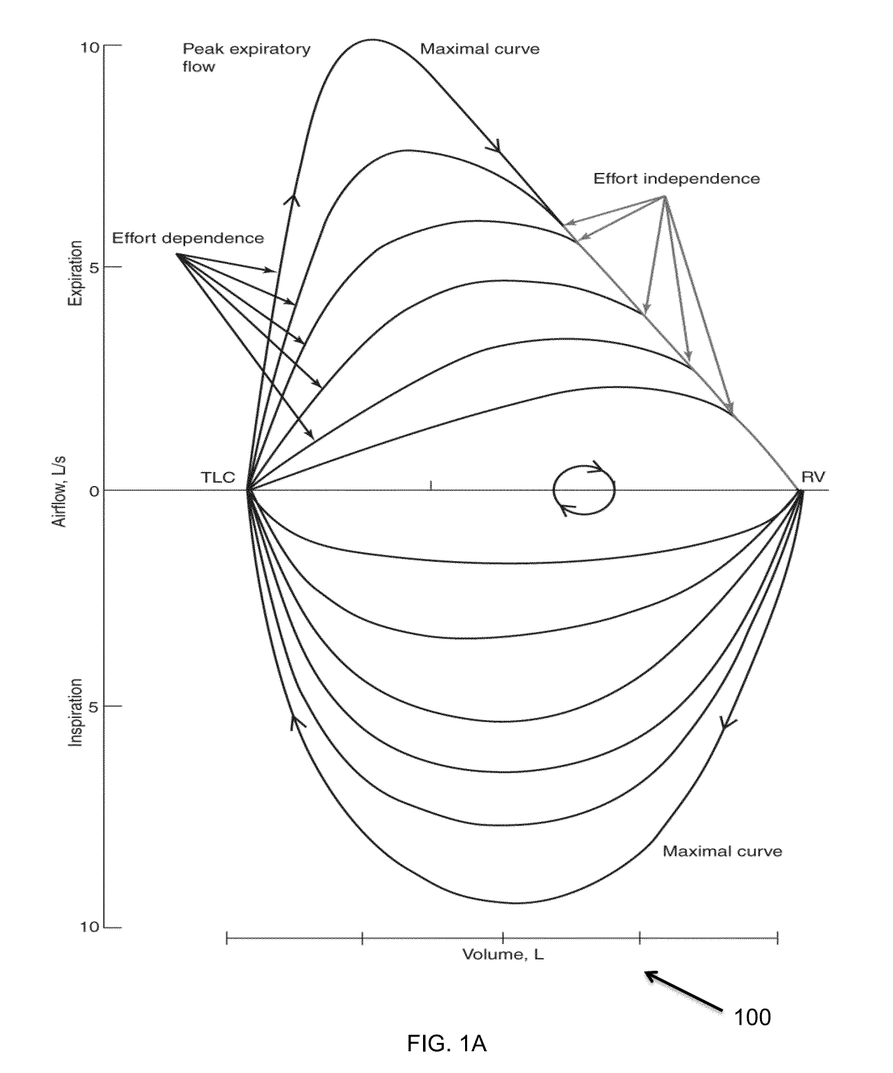

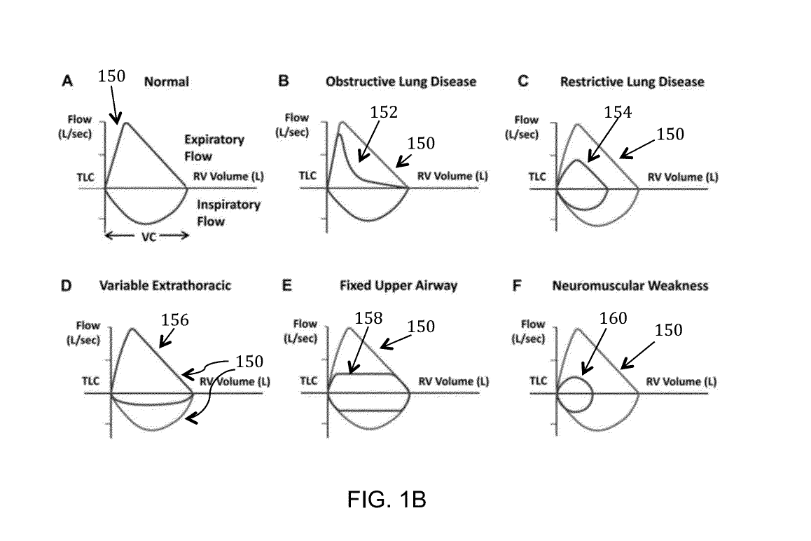

FIG. 1A is an illustration of an exemplary graph showing effect of a patient's effort on measurement of patient's respiratory system. In many currently available solutions for measuring respiratory systems, results may vary based on amount of patient effort. As such, some of these solutions may produce unreliable results.

FIG. 1B is an illustration of an exemplary graph showing differences between results of healthy patients and patients having various disorders as shown in separate graphs. Graphed results 150 may be produced by a typical normal healthy individual. Graphed results 152, 154, 156, 158 and 160 may be produced by typical individuals having the various indicated respiratory disorders. Point TLC on the x-axis represents total lung capacity, which is the maximum volume of air the lungs can hold within when fully expanded. Point RV on the x-axis represents the residual volume of air reminding/trapped in the lungs after a maximum effort to empty the lungs has been made. Those skilled in the field may know that there are various ways to measure TLC and RV such as, but not limited to, nitrogen washout technique or plethysmograph. Once TLC and RV values have been obtained, spirometer may be used to construct and plot the curves shown in FIG. 1B and fit the curves in between TLC (left side) and RV (right side) points on the x-axis. FIG. 1B illustrates the importance of the shape of graph produced by individuals for comparison with the typical normal healthy person. FIG. 1A shows the highly effort dependence of graph and how it may result in inaccurate diagnostic if a patient does not fully cooperate. As mentioned previously, Tiffeneau-Pinelli index value or FEV1/FVC is gold standard to diagnose respiratory disorders. Referring to FIG. 1A, TLC and RV may be measured separately and marked on the x-axis. The respiratory effort may then be fitted within these two points. The test starts at point TLC on the graph on the x-axis, where the patient has inhaled as much air possible to fully expand the lungs before the test starts. The test begins when the patient expire/empty their lungs as quick as they can, stopping only when it is not physically possible to blow/expire anymore air out of their lungs. The test typically ends here at point RV on the x-axis. Although the time component is not shown in FIG. 1A, and x-axis has the unit volume in liters, it may be assumed that values in the x-axis are directly correlated with time in seconds. Point TLC is 0 seconds representing the start of experiment, and point RV is however long/many seconds it takes to fully empty the lungs, representing the end of test. Y-axis represents the airflow in Liters per seconds. Positive Y values indicate positive flow/during expiration and negative values represent negative flow during inspiration, although the inspiration phase is shown in FIG. 1A, it is not conventionally used to calculate FEV1/FVC index value. On the top part of the graph during expiration, the six curves illustrate six different attempts with varying efforts to expire air. The top most graph, having the highest peak flow rate represents maximum effort put in by the patient, which is what's needed to get an accurate FEV1/FVC value, and the lower graphs there on illustrate various curves as the patient puts less and less effort during the test, lowest most graph representing least effort put by the patient. FEV1 can be measured by calculating the area under the expiratory curve within the first second [L/s*s=L]. It is intuitive and can also be seen on the graph, the less effort patient puts into the test, the lower airflow and subsequently reduced air volume will be expired within the first second of expiration. Reduced value of FEV1 will significantly change the calculated index value FEV1/FVC, hence compromise the accuracy of calculated index value that may be used to diagnose respiratory disorders.

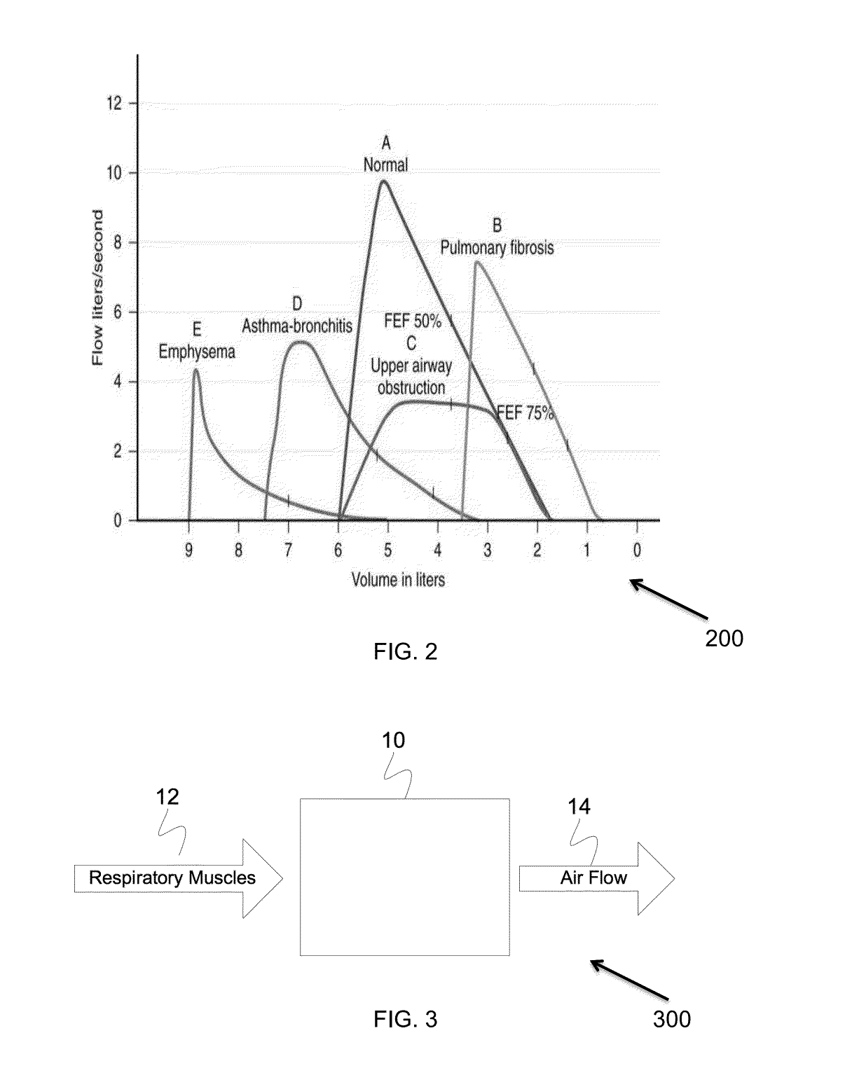

FIG. 2 is an illustration of an exemplary graph showing differences between results of healthy patients and patients having various disorders. This figure is similar to FIGS. 1A and 1B, having similar axis, however unlike FIG. 1B it illustrates various respiratory conditions in one graph. Graph A represents a normal healthy individual. The FEV1/FVC test starts at TLC, which is 6 liters in a healthy individual (graph A), 9 liters in patient with emphysema (graph E) and 3.5 liters in patient with pulmonary fibrosis (graph B) and so on. The test ends at RV point which is less than 2 L for healthy individual (graph A), 5 L for patient with emphysema (graph E) and less than 1 L for patient with pulmonary fibrosis. The y-axis represents the airflow speed in liters/second during the expiration attempt. These graphs have been constructed by statistical calculations and averaging the general population. Most of respiratory disorders have a long course of action. For example it could take as long as ten to twenty years, maybe even longer, for a patient that smokes to develop emphysema, during that time period patients FEV1/FVC curve will slowly shift from graph A, to graph E. Problem with current method is that it can only statistically differentiate curves A and E, when the patient has advanced well into their disease process, in this case emphysema. Many embodiments of the present invention directly compares respiratory systems input/muscles and output/airflow, mitigating the need to rely on statistical values as the respiratory systems input and output directly correlate with each other and depend on each individuals respiratory systems condition, independent of others in the population. For example, in a patient with emphysema, the lungs deteriorate, reducing the elasticity of lungs, hence respiratory muscles have to work less to expand the lungs. Similarly in a patient with pulmonary fibrosis, the lungs elasticity increases, hence the muscles have to work harder to expand the lungs. Therefore respiratory muscles activity is an important component to correlate and use in conjunction with airflow/output produced by the respiratory system.

FIG. 3 is an illustration of an exemplary system in which a respiratory unit receives input and produces output, in accordance with an embodiment of the present invention. Those having ordinary skill in the art will recognize that a person's respiratory system may behave in accordance with first law of thermodynamics, which states that energy cannot be generated or destroyed, but only changes form. Energy may transfer from chemical to mechanical or electrical, and energy may transform and be stored as potential to be used later or dissipate in a form of work such as heat or moving a mass. In the present embodiment, a respiration system 10 may receive input 12 and produce output 14. In some instances, respiration system 10 may include, without limitation, lungs and respiratory airways. In many instances, respiration system 10 may have unique physical properties that may, when altered, acutely and/or chronically result in respiratory problems, as seen in various pathological diseases. Respiration system's 10 physical properties may include, without limitation, elasticity of lungs as affected by number and layout of elastic fibers, total respiratory luminal area available for conducting air into and out of lungs, total vascular luminal area available for perfusing blood to and from lungs, total surface area available for gas exchange between alveoli and blood as well as surface thickness of barrier that may limit diffusion of gas between alveoli and blood. Changes in respiration system's 10 physical properties may disrupt physiological homeostasis, which may be sensed by central nervous system (CNS). To restore homeostasis, CNS may consciously or unconsciously try to compensate for the changes through different mechanisms such as, without limitation, changing respiratory pattern, rate of respiration, duration of respiration, depth of respiration, and constriction or dilation of blood vessels in lungs shunting blood to different parts of lung. Other pathological changes due to different diseases or harmful substances like smoking may also affect lungs' physical properties in chronic case. In acute cases, like pneumonia, lungs may fill with inflammatory cells, debris, and fluids that may produce a barrier/block diffusion of gases between blood and air within lungs. In some instances, input 12 may be provided by respiratory muscles to expand and/or compress lungs during inspiration and/or expiration, respectively. In healthy individuals, during normal expiration, most work may be done by utilizing potential energy stored in elastic tissues of lungs that may have previously been stretched during inspiration. During inspiration, depending on physical properties of respiration system 10, energy/work applied to system through respiratory muscles as a form of input may have two effects. A portion of energy/work may transform to and cause physical work to expand/stretch lungs and accompanying airways and blood vessels, which may in turn create negative pressure inside lungs and may suction air into lungs. A remaining portion may be stored as potential energy in elastic tissues of lungs, which may later be utilized during expiration phase. In addition to energy applied by respiratory muscles during expiration, potential energy that may have previously been stored in lung's elastic tissues may be utilized to restore stretched lungs or even compress lungs further, which may create positive pressure and pushing air out of lungs. Information as to which portion of energy is dissipated in a system to do work and what portion is stored in elastic tissues may directly relate to condition of lungs, pathological state, and restrictive versus obstructive respiratory disorder and may be formulated by comparing input 12 and output 14 of a system. In some instances, output 14 may be flow of air that may enter and leave lungs during inspiration and expiration, which may be used to compare with input 12 to predict properties of respiration system 10.

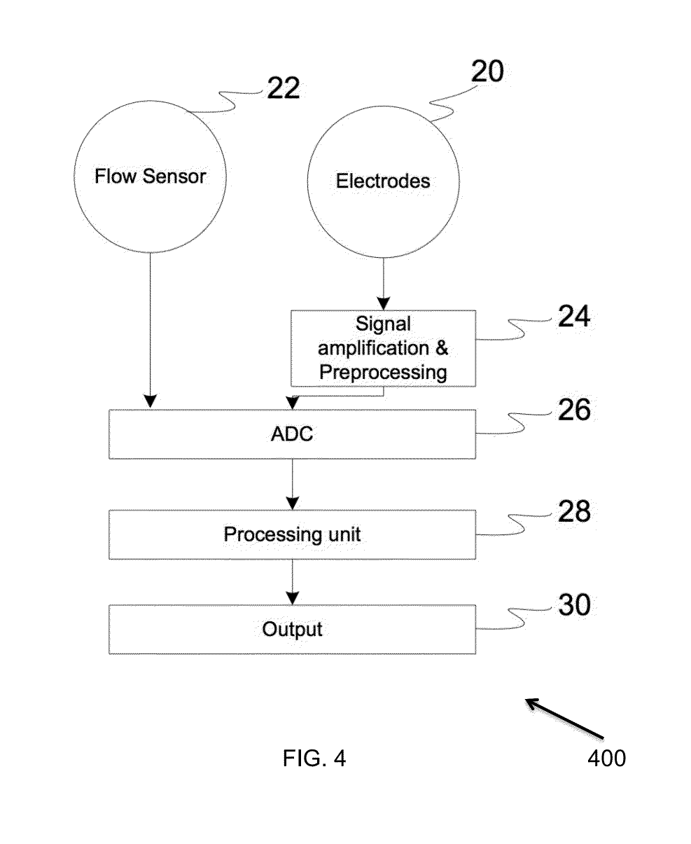

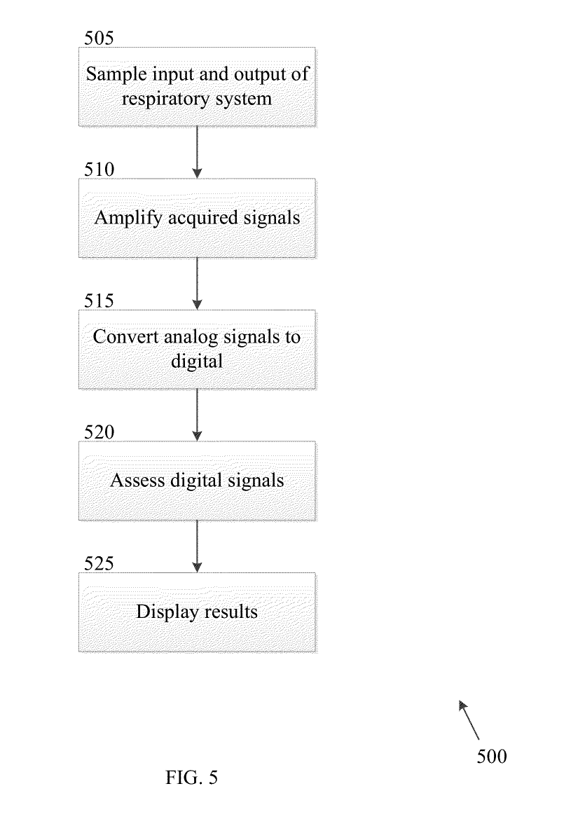

FIG. 4 is an illustration of an exemplary system for assessing respiratory system condition, in accordance with an embodiment of the present invention. In the present embodiment, electrodes 20 and flow sensor 22 may be used for measuring and may correspond to respiratory system input 12 and output 14, respectively. In some embodiments, electrodes 20 may be non-invasive sensors which may be used in electromyography to measure electrical activities of muscles. In some of these embodiments, electrodes 20 may be surface electrodes due to non-invasive characteristics. However, in other embodiments, invasive, needle-shapes electrodes may also be used. As a non-limiting example, invasive electrodes may provide a more accurate signal with better signal to noise ration as they may be inserted/targeted directly to each specific muscle by penetrating through the skin, however may not be preferred as it is uncomfortable for the patient and adds additional risks such as infection at the site of insertion. Some available scientific literature may demonstrate that measuring respiratory muscle electrical activities using surface electrodes may provide accurate electromyographs. In some embodiments, flow sensor 22 may be a simple mass flow sensor/transducer that may measure airflow into and out of lungs. As a non-limiting example, these sensors typically may have a built in circuit that amplifies the signal to a readable/desirable range, otherwise the amplification may be implemented as needed. In the present embodiment, preprocessing/amplification 24 may be an electrical circuit that may amplify and/or condition acquired signals. In a non-limiting example, signal conditioning goals may include, without limitation, selecting optimal frequency range for electromyography by applying high pass and low pass filters, removing unwanted noise from signals such as power line 60 Hz noise and heart electrical activity and/or inverting signal to convert negative amplitude signals to positive amplitude to be read by an analog to digital converter (ADC) 26. In the present embodiment, ADC 26 may be suitable for converting analog signals obtained into digital signals that may be read by a processing unit 28. In some embodiments, processing unit 28 may be a computer connected to a data acquisition module (DAQ) or an independent microcontroller. In many embodiments, processing unit 28 may serve different purposes such as, without limitation, further conditioning of signal digitally if needed, performing signal manipulations and/or calculations such as averaging mean amplitude/power of signals, taking integral or derivative of signal, calculating frequency response/spectrum, splitting signals according to stages of respiration cycle (inspiration vs. expiration), and converting obtained data into forms that may easily be interoperated by non-experts in field such as simple charts and tables. In some instances, these functions may allow a device to be used by non-expert users such as any healthcare staff with least knowledge of field. In some embodiments, processing unit 28 may implement algorithms to recognize patterns and/or suggest disorders based on signals obtained or sound an alarm in settings of emergency situation to notify for help. In a non-limiting example, if a patient is not breathing and/or may go into respiratory distress, processing unit 28 may sound an alarm. In the present embodiment, output 30 may be a screen or other means connected to a computer and/or microcontroller to accept and display results.

FIG. 5 is an illustration of an exemplary method for assessing respiratory system condition, in accordance with an embodiment of the present invention. In the present embodiment, a system may sample input 12 and output 14 of a respiratory system 10 in a step 505. In some embodiments, system may sample input 12 using electrodes 20. In some of these embodiments, electrodes 20 may be non-invasive. In other embodiments, electrodes 20 may be invasive. In a non-limiting example, needle-shaped electrodes 20 may be used. In many embodiments, electrodes 20 may measure electrical activities of muscles. In some embodiments, system may sample output 14 using a flow sensor 22. In some of these embodiments, flow sensor 22 may be a simple mass flow sensor/transducer that may measure airflow into and/or out of lungs. In the present embodiment, system may amplify and/or condition acquired signals in a step 510. In some embodiments, system may use one or more electrical circuits to amplify and/or condition acquired signals. In a non-limiting example, system may amplify measured electrical activities of muscles received from electrodes 20. In some embodiments, signal conditioning goals may include, without limitation, selecting an optimal frequency range for electromyography, removing unwanted noise from signals, and/or inverting signals to convert negative amplitude signals to positive amplitude. In the present embodiment, system may convert analog signals into digital signals in a step 515. In some embodiments, system may use an ADC 26 to convert analog signals into digital signals. In some of these embodiments, ADC 26 may receive positive amplitude signals from amplifier/conditioner 24. In other embodiments, ADC 26 may receive positive and negative amplitude signals from amplifier/conditioner 24. In the present embodiment, system may assess digital signals in a step 520. In some embodiments, a processing unit 28 may perform assessment of digital signals. In some of these embodiments, assessment may include, without limitation, further conditioning of signals, performing signal manipulations and/or calculations, computing integrals and/or derivatives of signals, calculating frequency response/spectrum, splitting signals according to stages of respiration cycle, and converting obtained data into forms that can easily be interoperated by non-experts. In the present embodiment, system may display results in a step 525. In some embodiments, results may be displayed to a computer screen.

It will be apparent to those skilled in the art that various modifications/rearrangement, additions/removals of certain steps may be made to the above-described exemplary embodiments of the present invention without departing from the scope of the invention. Thus, it is intended that the present invention covers all such modifications provided they come within the scope and spirit of appended claims and their equivalents. Examples of such additions may include, without limitation, changing/modifying/adding/removing steps after acquiring signals from electromyogram and flow transducer. In a non-limiting example, not doing any pre- or post-conditioning of data or just acquiring and displaying raw signals may be exemplary modifications. In another non-limiting example one may remove steps 515 and 520, directly feeding the sampled/live analog signals collected to a means of physical display such as printing on a paper, without the need to digitally represent and store the signals. In another non-limiting example, one may use invasive needles or other types of electrodes 20 instead of using non-invasive electromyography electrodes 20, this may result in a stronger signal and allow to remove step 510, signal amplification. In still another non-limiting example, instead of using a flow transducer, one may use pressure or other types of transducers that may still acquire respiratory “output” as air entering and exiting lungs. In another non-limiting example, one may implement/add other sensors to make a device more useful such as, without limitation, an oxygen sensor to measure oxygen concentration of air breathing, LEDs+photodiode—that may functions as a pulse oximetry to measure patients oxygen saturation, CO2 sensors to measure patients carbon dioxide concentration, infra-red beam and detector to function as capnography for measuring CO2 partial pressure of respiratory gases, nitrogen sensor for measuring lungs dead space, helium sensor for measuring lungs functional residual capacity, CO+methane or other gas sensors for measuring lungs diffusing capacity, etc.

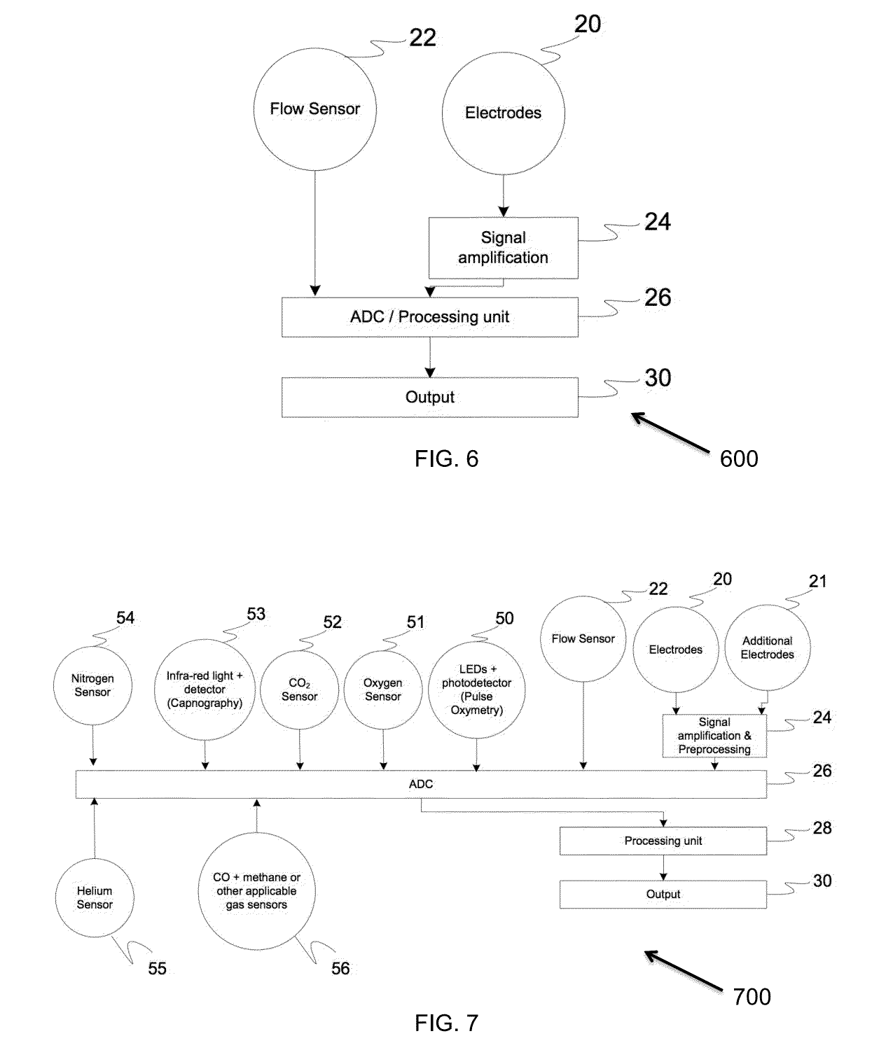

FIG. 6 is an illustration of an exemplary simplified system for assessing respiratory system condition, in accordance with an embodiment of the present invention. In the present embodiment, system may acquire input 20 and output 22 of a respiratory system 10. Further, in the present embodiment, system may display 30 data. However, in some embodiments, system may display data without any processing or manipulation of the data. In the present embodiment, system may amplify signals using an ADC 26. However, in some embodiments, if ADC/processing unit 26 may be capable of accurately reading weak signals in micro or millivolt ranges, system may not need to amplify signals. Similarly if invasive electrodes are used instead of non-invasive surface electrodes, obtained signal may be strong enough and not need to be amplified. In some of these embodiments, ADC/processing unit 26 may be replaced by a microcontroller or other suitable device that may have a built-in ADC which may be capable of connecting to a screen to display signals. Some of these embodiments may allow input 20 and output 22 signals to be acquired in a same time domain and hence may allow for comparison of the signals with respect to each other.

FIG. 7 is an illustration of an exemplary system for assessing respiratory system condition, having additional components, in accordance with an embodiment of the present invention. In some embodiments, a system may implement additional sensors to system shown in FIG. 4. In some of these embodiments, additional sensors may provide additional information about respiratory system. In the present embodiment, system may implement additional electrodes 21. In some embodiment, additional electrodes 21 may be suitable to measure cardiac muscle activity. In the present embodiment, system may implement LEDs+photo detector sensor, to function as a pulse oximetry 50. In some embodiments, photo detector sensor 50 may be suitable for measuring blood oxygen saturation levels. In the present embodiment, system may implement an oxygen sensor 51. In some embodiments, oxygen sensor 51 may be suitable for measuring oxygen concentration of breathing air and/or of blood. In the present embodiment, system may implement a CO2 sensor 52. In some embodiments, CO2 sensor 52 may be suitable for measuring CO2 concentration of breathing air within the lungs and/or of blood. In the present embodiment, system may implement an infra-red light and a detector 53. In some embodiment infra-red sensor 53 may be suitable for measuring partial pressure of CO2 within breathing gases, as used in capnography. In present embodiments, system may implement a nitrogen sensor 54. In some embodiments, nitrogen sensor 54 may be suitable to perform nitrogen washout test for measuring dead space in lungs. In present embodiments, system may implement a helium sensor 55. In some embodiment, helium sensor 55 may be suitable to perform helium dilution technique for measuring lungs functional residual capacity. In present embodiments, system may implement a CO+methane or other applicable gases 56. In some embodiment, CO+methane or other applicable gases 56 may be suitable to measure lungs diffusing capacity. In other embodiments, any form of sensor or apparatus that may collect useful information about, or related to a state and/or functionality of a respiratory system may be added to embodiments of the current invention.

In one embodiment, the DAQ may be a USB 1608G-series DAQ by Measurement Computing. In at least one embodiment, DAQ may be used in an 8 differential analog input configuration, in which one channel may be dedicated for airflow transducer and seven channels may be connected to a preprocessing/amplification circuit for seven bipolar electrode leads plus one ground, totaling fifteen leads. In a non-limiting example, an airflow transducer may be a Honeywell AWM730P1.

In one or more embodiments, signal amplification may be performed using INA129 precision, low power instrumentational amplifiers by Texas Instruments, with a variable resistor for flexible amplification. In some of these embodiments, TLE207 excaliber low-noise high-speed JFET-INPUT operational amplifiers may be used to further amplify and/or high pass signals using capacitors. In one or more embodiments, low cutoff frequency may be set at 100 Hz, which may provide a convenient method to remove 60 Hz line noise as well as noise from cardiac muscle activity. In some of these embodiments, the method may remove some of respiratory muscle activity signals below 100 Hz, but may receive substantially strong enough signals from respiratory muscles above 100 Hz as DAQ module may allow for sample data at 2000 Hz and above. In one of these embodiments, a frequency of 2000 Hz may provide an effective signal-to-noise ratio using embodiment circuit.

Some embodiments may be powered with a KMT15-51515 power supply made by TDK-Lamba, which is a medical grade/certified power supply, to maximize safety. In some of these embodiments, power supply may be connected to a wall outlet using an FN9222B medical grade IEC Inlet Filter.

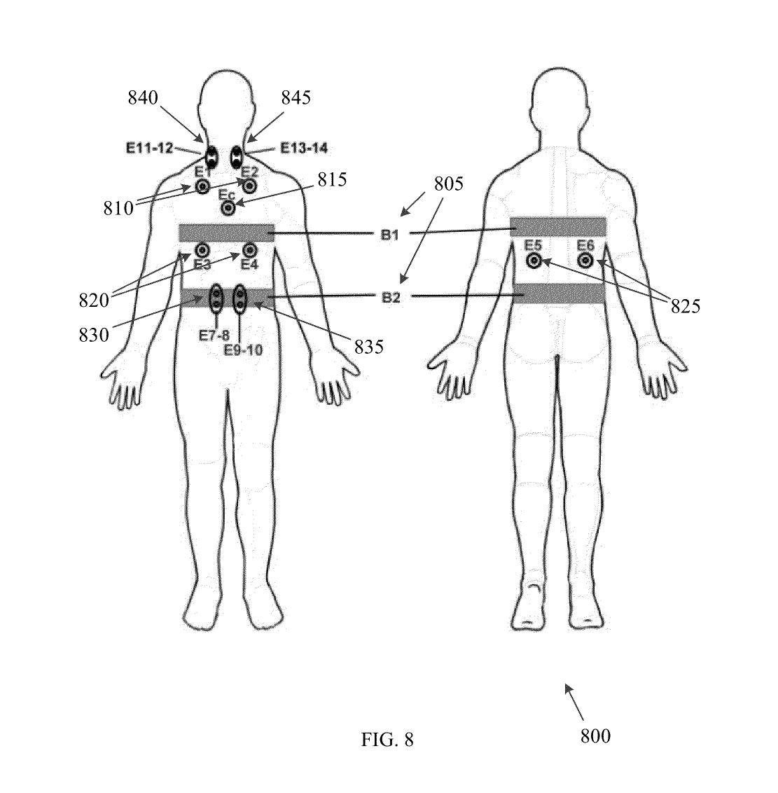

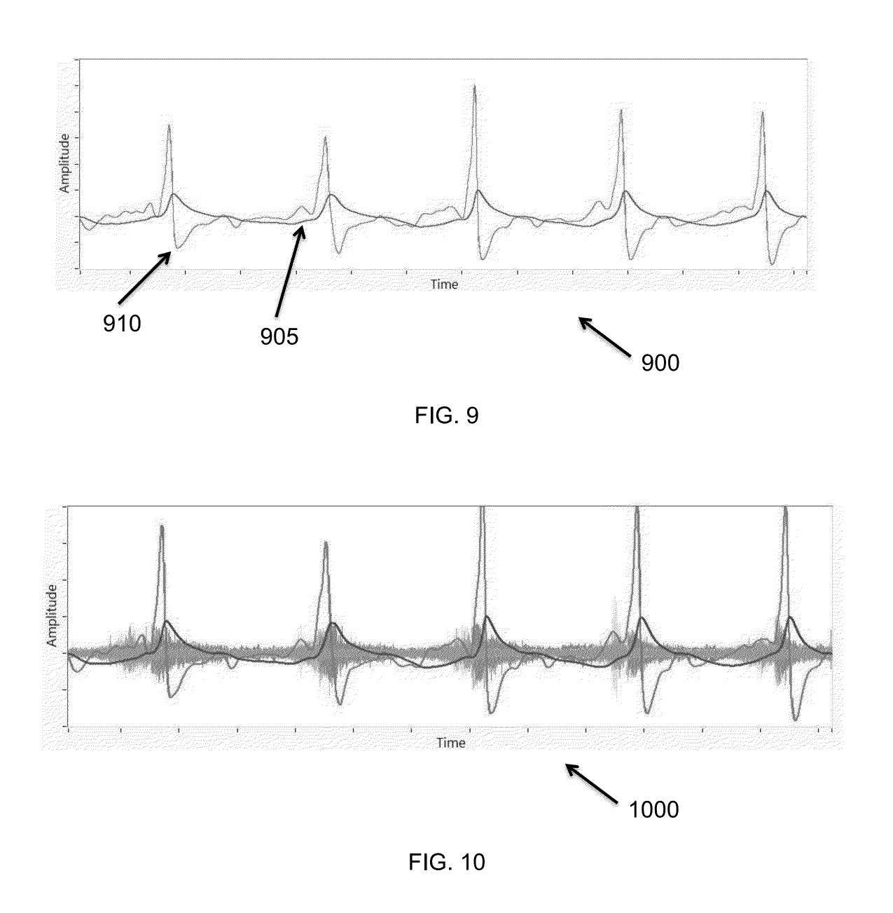

FIG. 8 is an illustration of exemplary placement of electrodes 20, in accordance with an embodiment of the present invention. Some embodiments may utilize approximately seven bipolar electrodes 20 to capture important muscles involved in respiration, and electrodes 20 may be arranged in a suitable configuration. In the present embodiment, system may include, without limitation: two magnetic respiration bands 805; common electrode 810; intercostal electrodes 815; frontal diaphragm electrodes 820; dorsal diaphragmatic electrodes 825; right 830 and left 835 abdominal electrodes; and right 840 and left 845 scalene muscle electrodes. In some instances, a system in which bilaterally widely separated electrodes placed in costal spaces below costal margin may obtain high-quality EMG recording, which may be minimally disturbed by unwanted external factors. In some embodiments, one pair of electrodes may be placed bilaterally at costal margin in nipple line, one pair bilaterally on back at level of diaphragm, one pair in second intercostal spaces one electrode left and one right, with 3 cm parasternal, and bipolar electrodes left and right on neck over Sc. Other embodiments may allow for any number of electrodes, suitable arrangements and/or techniques to capture respiratory muscles activity. In some embodiments, one may use unipolar electrodes and/or add more ground leads placed closely to each muscle of interest to get a more accurate signal. In other embodiments, one may use a single bipolar electrode to only capture one respiratory muscle, e.g. diaphragm muscle, which may be most important muscle involved in respiration.

In some embodiments, Labview software may be used to read data from DAQ module for data manipulation, calculations/analysis, graphical representation, etc. However, in other embodiments, Matlab or any other software may be used, or even a preprogrammed microcontroller.

FIG. 9 is an illustration of an exemplary graph measuring airflow of a respiratory system, in accordance with an embodiment of the present invention. In the present embodiment, airflow 905 may be measured in liters/second, and may be plotted over five respiratory cycles. In other embodiments, any number of respiratory cycles may be plotted. In some embodiments, a transducer may be suitable for measuring airflow. In the present embodiment, positive values may indicate air being expired and negative values may indicate air being inspired. In some embodiments, one may use an absolute value of a graph to reflect both inspiration and expiration in a positive axis, then one may integrate with respect to x axis/time in seconds to find area under the graph which may represent total volume of air exchanged/displaced in liters during respiratory cycles. In the present embodiment, graph 900 may also represent acceleration 910. Acceleration (a) is directly proportional to applied force (F), F=m*a. In some instances, F may be a force applied by respiratory muscles to lungs, and lungs may then transfer the force and apply it on mass (m) molecules of air within lungs to accelerate (a) them out of the lungs or create negative pressure and suction air in. In some embodiments, one may calculate an instantaneous derivative of volume (L/S) graph with respect to x-axis/time in seconds to plot instantaneous acceleration 910. In some of these embodiments, one may invert negative portions of acceleration 910 graph to reflect in positive axis for time interval integration of cycles. In many instances, a closed system in a closed room in which no air may be added or removed may create an assumption of constant mass/air (m) within system boundary for force equation (F=m*a), meaning respiratory system may simply be moving/displacing air within closed system. In some embodiments, one may integrate force equation with respect to time and calculate work done by system by integrating acceleration graph 910 that may represent work done by system to create “changing acceleration”. In some of these embodiments, work done by a system to move air may be abbreviated as WS, total work applied to the system by respiratory muscles may be abbreviated as WA, and work dissipated may be abbreviated as WD. Further, in some of these embodiments, using first law of thermodynamics, WA=WS+WD. In many instances, time interval integration of electromyogram may provide a good estimate of work done by muscles, hence providing a calculation for WA. In some instances, it may be difficult to measure WD, and one may not be able to assume the same units (e.g. joules) for all the variables, hence one may not simply rearrange the equation to solve for variables. However, in some instances, one may assume a fraction (f) of WA may be used to do work within the system/displace air, WS, and the remaining fraction of WA may be dissipated within the system depending on condition of lungs/system, hence one may conclude WA=f*WS, or f=WA/WS. This f value may provide important information about the condition of respiratory system, as the change in f value may be a sensitive variable for distinguishing between healthy individuals, obstructive and restrictive respiratory disorders.

FIG. 10 is an illustration of an exemplary graph of respiratory cycles showing volume flow, acceleration, and all EMG amplitudes in one graph, in accordance with an embodiment of the present invention.

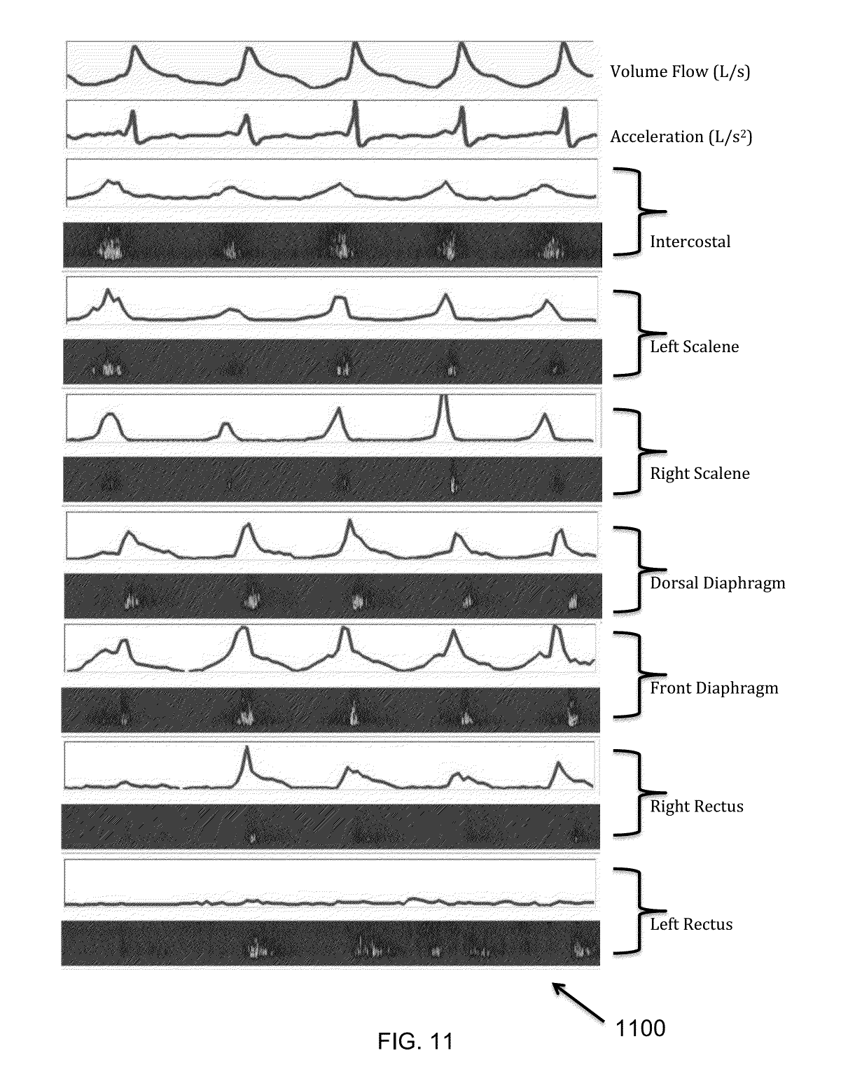

FIG. 11 is an illustration of an exemplary graph of respiratory cycles showing volume flow, acceleration, and EMG amplitudes as wells as using a short-time Fourier transform to show perspective time-frequency response associated with each muscle, in accordance with an embodiment of the present invention.

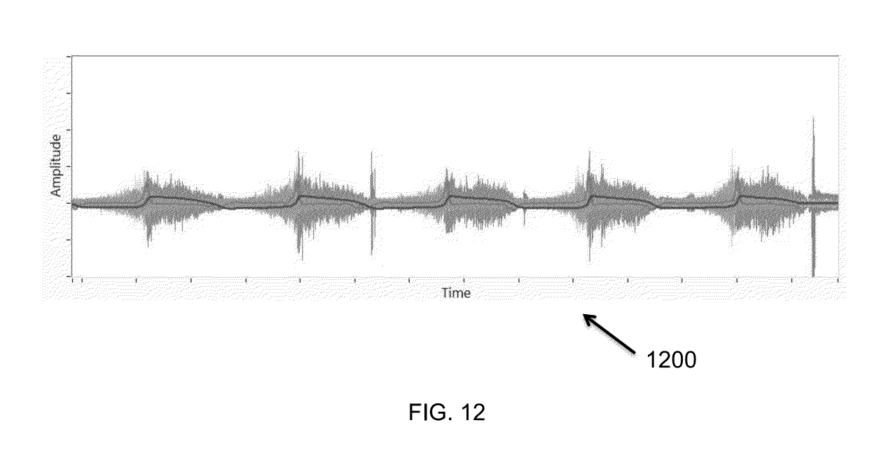

FIG. 12 is an illustration of an exemplary graph of respiratory cycles showing volume flow, acceleration, and all EMG amplitudes in one graph, and in which respiratory cycles have been measured in a simulated obstructive condition (valve partially closed), in accordance with an embodiment of the present invention.

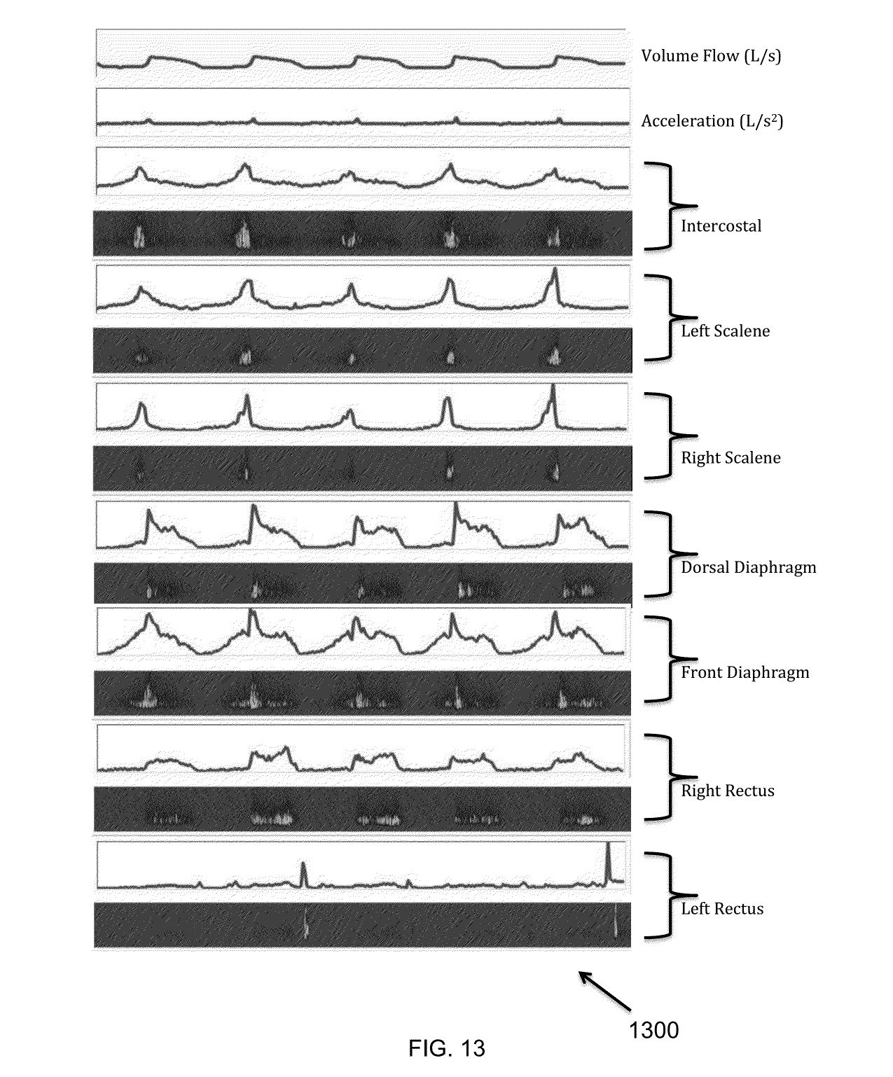

FIG. 13 is an illustration of an exemplary graph of respiratory cycles showing volume flow, acceleration, and EMG amplitudes as wells as using a short-time Fourier transform to show perspective time-frequency response associated with each muscle, and in which respiratory cycles have been measured in a simulated obstructive condition (valve partially closed) in accordance with an embodiment of the present invention.

In the present embodiments, comparison of FIG. 10 to FIG. 12 and comparison of FIG. 11 to FIG. 13 may illustrate that respiratory muscles may work harder in an obstructive condition, and a greater amplitude and frequency response while peak volume flow and acceleration curve may be reduced.

Some embodiments of the present invention may be used in a variety of different environments and settings. One or more embodiments may be built as stand-alone devices/units that may be preprogrammed using a microcontroller with desired/preferred ways of data analysis and presentation. Some embodiments may simply collect data and display them on a screen, or add more flexibility as needed in research. Many embodiments may be used in different settings, such as, without limitation, screening patients, making diagnostic calls, monitoring progress of disease and response to certain treatments, monitoring patients' vital signs during a surgery, or for sounding an alarm when patients may go into respiratory distress in an emergency situation or during a surgery. Some embodiments may be suitable for providing education about respiratory systems by allowing for study of different respiratory muscles and their roles at different phases of respiratory cycle, and for studying different patterns of breathing and associating the patterns with usage of various respiratory muscles.

At least one embodiment of the present invention may be implemented in any emergency setting/situation/device or during a surgery. In a non-limiting example, embodiments may be used in ambulances or hospital emergency or surgery rooms to monitor patient breathing or other important information related to respiratory system such as patient airway, CO2 concentration, breathing air or blood oxygen concentration, or blood oxygen saturation, or other cardiovascular parameters as related to tissue oxygenation.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

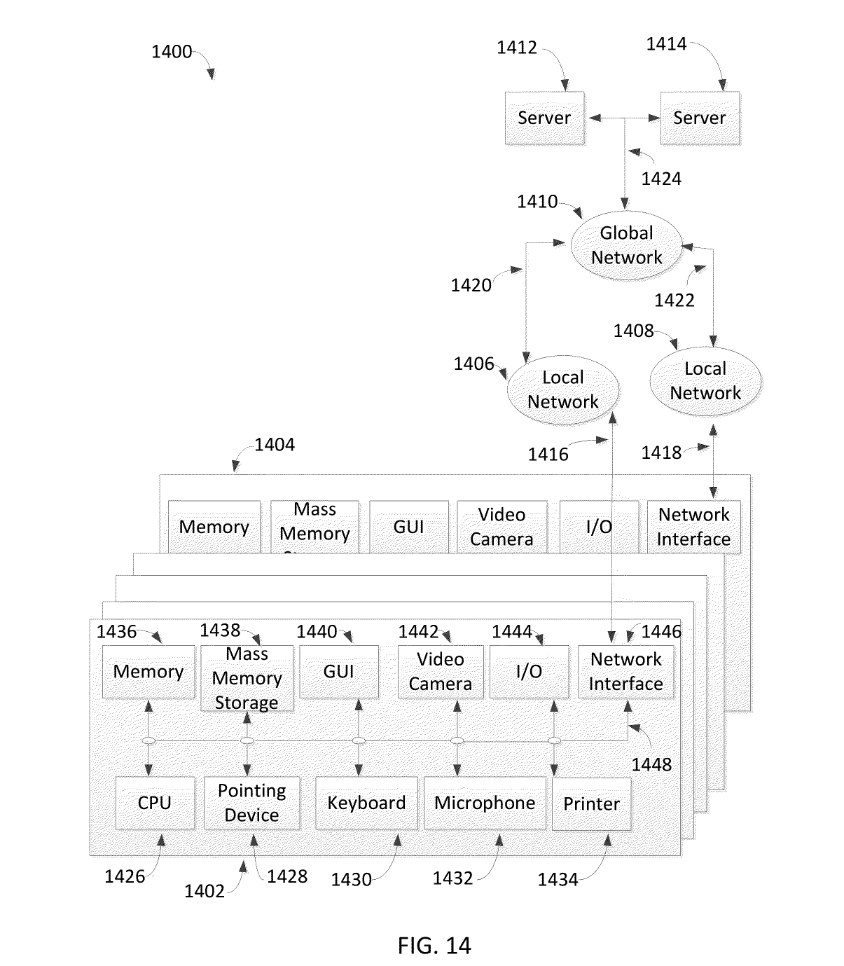

FIG. 14 is a block diagram depicting an exemplary client/server system which may be used by an exemplary web-enabled/networked embodiment of the present invention.

A communication system 1400 includes a multiplicity of clients with a sampling of clients denoted as a client 1402 and a client 1404, a multiplicity of local networks with a sampling of networks denoted as a local network 1406 and a local network 1408, a global network 1410 and a multiplicity of servers with a sampling of servers denoted as a server 1412 and a server 1414.

Client 1402 may communicate bi-directionally with local network 1406 via a communication channel 1416. Client 1404 may communicate bi-directionally with local network 1408 via a communication channel 1418. Local network 1406 may communicate bi-directionally with global network 1410 via a communication channel 1420. Local network 1408 may communicate bi-directionally with global network 1410 via a communication channel 1422. Global network 1410 may communicate bi-directionally with server 1412 and server 1414 via a communication channel 1424. Server 1412 and server 1414 may communicate bi-directionally with each other via communication channel 1424. Furthermore, clients 1402, 1404, local networks 1406, 1408, global network 1410 and servers 1412, 1414 may each communicate bi-directionally with each other.

In one embodiment, global network 1410 may operate as the Internet. It will be understood by those skilled in the art that communication system 1400 may take many different forms. Non-limiting examples of forms for communication system 1400 include local area networks (LANs), wide area networks (WANs), wired telephone networks, wireless networks, or any other network supporting data communication between respective entities.

Clients 1402 and 1404 may take many different forms. Non-limiting examples of clients 1402 and 1404 include personal computers, personal digital assistants (PDAs), cellular phones and smartphones.

Client 1402 includes a CPU 1426, a pointing device 1428, a keyboard 1430, a microphone 1432, a printer 1434, a memory 1436, a mass memory storage 1438, a GUI 1440, a video camera 1442, an input/output interface 1444 and a network interface 1446.

CPU 1426, pointing device 1428, keyboard 1430, microphone 1432, printer 1434, memory 1436, mass memory storage 1438, GUI 1440, video camera 1442, input/output interface 1444 and network interface 1446 may communicate in a unidirectional manner or a bi-directional manner with each other via a communication channel 1448. Communication channel 1448 may be configured as a single communication channel or a multiplicity of communication channels.

CPU 1426 may be comprised of a single processor or multiple processors. CPU 1426 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 1436 is used typically to transfer data and instructions to CPU 1426 in a bi-directional manner. Memory 1436, as discussed previously, may include any suitable computer-readable media, intended for data storage, such as those described above excluding any wired or wireless transmissions unless specifically noted. Mass memory storage 1438 may also be coupled bi-directionally to CPU 1426 and provides additional data storage capacity and may include any of the computer-readable media described above. Mass memory storage 1438 may be used to store programs, data and the like and is typically a secondary storage medium such as a hard disk. It will be appreciated that the information retained within mass memory storage 1438, may, in appropriate cases, be incorporated in standard fashion as part of memory 1436 as virtual memory.

CPU 1426 may be coupled to GUI 1440. GUI 1440 enables a user to view the operation of computer operating system and software. CPU 1426 may be coupled to pointing device 1428. Non-limiting examples of pointing device 1428 include computer mouse, trackball and touchpad. Pointing device 1428 enables a user with the capability to maneuver a computer cursor about the viewing area of GUI 1440 and select areas or features in the viewing area of GUI 1440. CPU 1426 may be coupled to keyboard 1430. Keyboard 1430 enables a user with the capability to input alphanumeric textual information to CPU 1426. CPU 1426 may be coupled to microphone 1432. Microphone 1432 enables audio produced by a user to be recorded, processed and communicated by CPU 1426. CPU 1426 may be connected to printer 1434. Printer 1434 enables a user with the capability to print information to a sheet of paper. CPU 1426 may be connected to video camera 1442. Video camera 1442 enables video produced or captured by user to be recorded, processed and communicated by CPU 1426.

CPU 1426 may also be coupled to input/output interface 1444 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 1426 optionally may be coupled to network interface 1446 which enables communication with an external device such as a database or a computer or telecommunications or internet network using an external connection shown generally as communication channel 1416, which may be implemented as a hardwired or wireless communications link using suitable conventional technologies. With such a connection, CPU 1426 might receive information from the network, or might output information to a network in the course of performing the method steps described in the teachings of the present invention.

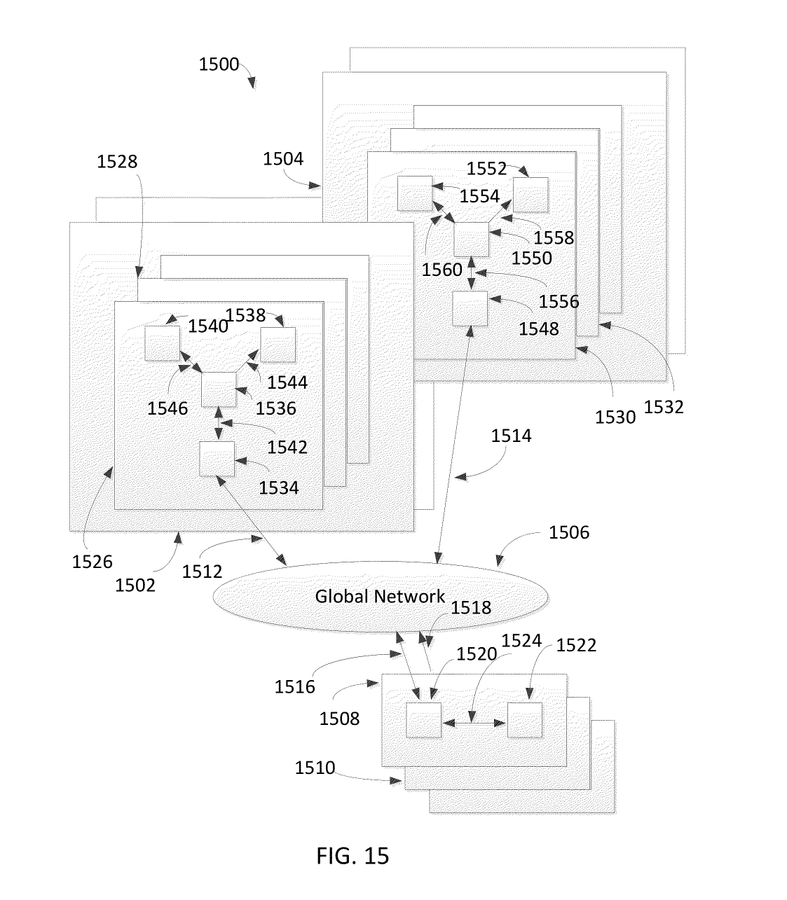

FIG. 15 illustrates a block diagram depicting a conventional client/server communication system.

A communication system 1500 includes a multiplicity of networked regions with a sampling of regions denoted as a network region 1502 and a network region 1504, a global network 1506 and a multiplicity of servers with a sampling of servers denoted as a server device 1508 and a server device 1510.

Network region 1502 and network region 1504 may operate to represent a network contained within a geographical area or region. Non-limiting examples of representations for the geographical areas for the networked regions may include postal zip codes, telephone area codes, states, counties, cities and countries. Elements within network region 1502 and 1504 may operate to communicate with external elements within other networked regions or within elements contained within the same network region.

In some implementations, global network 1506 may operate as the Internet. It will be understood by those skilled in the art that communication system 1500 may take many different forms. Non-limiting examples of forms for communication system 1500 include local area networks (LANs), wide area networks (WANs), wired telephone networks, cellular telephone networks or any other network supporting data communication between respective entities via hardwired or wireless communication networks. Global network 1506 may operate to transfer information between the various networked elements.

Server device 1508 and server device 1510 may operate to execute software instructions, store information, support database operations and communicate with other networked elements. Non-limiting examples of software and scripting languages which may be executed on server device 1508 and server device 1510 include C, C++, C# and Java.

Network region 1502 may operate to communicate bi-directionally with global network 1506 via a communication channel 1512. Network region 1504 may operate to communicate bi-directionally with global network 1506 via a communication channel 1514. Server device 1508 may operate to communicate bi-directionally with global network 1506 via a communication channel 1516. Server device 1510 may operate to communicate bi-directionally with global network 1506 via a communication channel 1518. Network region 1502 and 1504, global network 1506 and server devices 1508 and 1510 may operate to communicate with each other and with every other networked device located within communication system 1500.

Server device 1508 includes a networking device 1520 and a server 1522. Networking device 1520 may operate to communicate bi-directionally with global network 1506 via communication channel 1516 and with server 1522 via a communication channel 1524. Server 1522 may operate to execute software instructions and store information.

Network region 1502 includes a multiplicity of clients with a sampling denoted as a client 1526 and a client 1528. Client 1526 includes a networking device 1534, a processor 1536, a GUI 1538 and an interface device 1540. Non-limiting examples of devices for GUI 1538 include monitors, televisions, cellular telephones, smartphones and PDAs (Personal Digital Assistants). Non-limiting examples of interface device 1540 include pointing device, mouse, trackball, scanner and printer. Networking device 1534 may communicate bi-directionally with global network 1506 via communication channel 1512 and with processor 1536 via a communication channel 1542. GUI 1538 may receive information from processor 1536 via a communication channel 1544 for presentation to a user for viewing. Interface device 1540 may operate to send control information to processor 1536 and to receive information from processor 1536 via a communication channel 1546. Network region 1504 includes a multiplicity of clients with a sampling denoted as a client 1530 and a client 1532. Client 1530 includes a networking device 1548, a processor 1550, a GUI 1552 and an interface device 1554. Non-limiting examples of devices for GUI 1538 include monitors, televisions, cellular telephones, smartphones and PDAs (Personal Digital Assistants). Non-limiting examples of interface device 1540 include pointing devices, mousse, trackballs, scanners and printers. Networking device 1548 may communicate bi-directionally with global network 1506 via communication channel 1514 and with processor 1550 via a communication channel 1556. GUI 1552 may receive information from processor 1550 via a communication channel 1558 for presentation to a user for viewing. Interface device 1554 may operate to send control information to processor 1550 and to receive information from processor 1550 via a communication channel 1560.

For example, consider the case where a user interfacing with client 1526 may want to execute a networked application. A user may enter the IP (Internet Protocol) address for the networked application using interface device 1540. The IP address information may be communicated to processor 1536 via communication channel 1546. Processor 1536 may then communicate the IP address information to networking device 1534 via communication channel 1542. Networking device 1534 may then communicate the IP address information to global network 1506 via communication channel 1512. Global network 1506 may then communicate the IP address information to networking device 1520 of server device 1508 via communication channel 1516. Networking device 1520 may then communicate the IP address information to server 1522 via communication channel 1524. Server 1522 may receive the IP address information and after processing the IP address information may communicate return information to networking device 1520 via communication channel 1524. Networking device 1520 may communicate the return information to global network 1506 via communication channel 1516. Global network 1506 may communicate the return information to networking device 1534 via communication channel 1512. Networking device 1534 may communicate the return information to processor 1536 via communication channel 1542. Processor 1546 may communicate the return information to GUI 1538 via communication channel 1544. User may then view the return information on GUI 1538.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing respiratory system assessment according to the present invention will be apparent to those skilled in the art. Various aspects of the invention have been described above by way of illustration, and the specific embodiments disclosed are not intended to limit the invention to the particular forms disclosed. The particular implementation of the respiratory system assessment may vary depending upon the particular context or application. By way of example, and not limitation, the respiratory system assessment described in the foregoing were principally directed to input/output implementations; however, similar techniques may instead be applied to systems employing only input or output measurement, or performing measurement of any other aspect of respiratory system, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A system comprising:
at least one flow sensor being configured to sample a rate of airflow in a patients lungs during inspiration and/or expiration of the patient and to output a corresponding airflow signal;
at least one sensor being configured to sample respiratory muscles signals of the patient simultaneously with said sampling of said airflow and to output a corresponding activity signal; and
at least one display unit being configured to at least display said airflow signal and said activity signal simultaneously wherein a comparison of at least said airflow signal and said activity signal indicates physical properties of a respiratory system of the patient.

2. The system as recited in claim 1, further comprising a processing unit being configured to at least process said airflow signal to produce an instantaneous acceleration signal corresponding to said airflow signal for display by said display unit.

3. The system as recited in claim 2, in which said airflow signal, said activity signal, and said instantaneous acceleration signal are displayed superimposed.

4. The system as recited in claim 1, in which said processing unit is further configured to produce a Fourier transform of said activity signal for display by said display unit.

5. The system as recited in claim 1, further comprising an optical sensor for sampling pulse oximetry to produce a pulse oximetry signal for display.

6. The system as recited in claim 1, further comprising a carbon dioxide sensor for sampling carbon dioxide concentration of the patient to produce a carbon dioxide signal for display.

7. The system as recited in claim 1, further comprising an oxygen sensor for sampling oxygen concentration of the patient to produce an oxygen signal for display.

8. The system as recited in claim 1, in which said at least one sensor to sample respiratory muscles signals comprises a plurality of electrodes disposed on the patient.

9. The system as recited in claim 8, further comprising at least one amplifier being configured to amplify and filter outputs of said plurality of electrodes for display as a plurality of activity signals on said display unit.

10. A system comprising:
means for sampling a rate of airflow in a patients lungs during inspiration and/or expiration of the patient and for outputting a corresponding airflow signal;
means for sampling respiratory muscles signals of the patient simultaneously with said sampling of said airflow and for outputting a corresponding activity signal; and
means for displaying said airflow signal and said activity signal simultaneously wherein a comparison of at least said airflow signal and said activity signal indicates physical properties of a respiratory system of the patient.

11. The system as recited in claim 10, further comprising means for processing said airflow signal to produce an instantaneous acceleration signal corresponding to said airflow signal and for producing a Fourier transform of said activity signal for display by said display unit.

12. The system as recited in claim 10, further comprising means for sampling pulse oximetry to produce a pulse oximetry signal for display.

13. The system as recited in claim 10, further comprising means for sampling carbon dioxide concentration of the patient to produce a carbon dioxide signal for display.

14. The system as recited in claim 10, further comprising means for sampling oxygen concentration of the patient to produce an oxygen signal for display.

15. A method comprising the steps of:
sampling a rate of airflow in a patients lungs during inspiration and/or expiration of the patient and to output a corresponding airflow signal;
sampling respiratory muscles signals of the patient simultaneously with said sampling of said airflow and to output corresponding activity signals; and
displaying said airflow signal and said activity signals simultaneously and superimposed wherein a comparison of at least said airflow signal and said activity signals indicates physical properties of a respiratory system of the patient.

16. The method as recited in claim 15, further comprising the step of processing said airflow signal to produce an instantaneous acceleration signal corresponding to said airflow signal and to produce Fourier transforms of said activity signals for displaying.

17. The method as recited in claim 15, further comprising the step of sampling pulse oximetry for displaying.

18. The method as recited in claim 15, further comprising the step of sampling carbon dioxide concentration of the patient for displaying.

19. The method as recited in claim 15, further comprising the step of sampling oxygen concentration of the patient for displaying.

20. The method as recited in claim 15, further comprising the step of amplifying and filtering said activity signals.
